(19) United States
(12) Patent Application Publication
(10) Pub. No.: US 2026/0062361 A1
(43) Pub. Date: Mar. 5, 2026

(54) PROCESS FOR THE PREPARATION OF BIO-BASED OLEFINS FROM PRIMARY ALCOHOL

(71) Applicant: BASF SE, Ludwigshafen am Rhein (DE)

(72) Inventors: Jessica Nadine HAMANN, Ludwigshafen am Rhein (DE); Peter OECHSLE, Ludwigshafen am Rhein (DE); Joaquim Henrique TELES, Ludwigshafen am Rhein (DE); Bernhard BRUNNER, Ludwigshafen am Rhein (DE); Alois KINDLER, Ludwigshafen am Rhein (DE); Michael Ludwig LEJKOWSKI, Ludwigshafen am Rhein (DE)

(21) Appl. No.: 19/103,295

(22) PCT Filed: Aug. 15, 2023
(86) PCT No.: PCT/EP2023/072454

§ 371 Date: Feb. 12, 2025

(30) Foreign Application Priority Data
Aug. 17, 2022 (EP) 22190674.6

(51) Int. Cl. C07C 1/207

(52) U.S. Cl. CPC C07C 1/2078; C07C 2523/42; C07C 2531/30

(57) ABSTRACT

Propylene is an important industrial intermediate for the production of propylene oxide and polypropylene, while isobutylene is similarly widely used for the production of a variety of industrially important products, such as butyl rubber. Both of propylene and isobutylene are obtained through the catalytic or steam cracking of fossil feedstocks, and the development of a commercially viable process for the direct conversion of alcohol to either material would accordingly be of great interest as fossil resources are depleted and/or become more costly to use, especially in consideration of increased demand for both of propylene and isobutylene. The present invention relates to the process for preparing lower olefins from primary alcohols. More particularly the present invention relates to the process of conversion of primary alcohols to lower olefins wherein the primary alcohols are obtained from renewable sources.

### FIELD OF INVENTION

The present invention relates to the process for preparing lower olefins from primary alcohols. More particularly the present invention relates to the process of conversion of primary alcohol which has at least one hydrogen atom attached to the carbon atom adjacent to the one bearing the alcohol group to lower olefins wherein the primary alcohols are obtained from renewable sources.

### BACKGROUND

Biomass is considered as a CO₂ neutral energy carrier, and is one of the most abundant and renewable of natural resources. In recent years, both as a result of market conditions as well as in response to a variety of governmental initiatives and mandates, biomass transformation to produce biofuel is has attracted significant effort and investment. With the increased availability and reduced cost of bioethanol, opportunities have been explored to use bioethanol not just as a biofuel but as a feedstock for making a variety of renewable source-derived chemicals.

The most commercially advanced initiative has been the dehydration of bioethanol to produce ethylene, though the conversions of bioethanol to propylene and isobutylene have also been studied. Propylene is an important industrial intermediate for the production of propylene oxide and polypropylene, while isobutylene is similarly widely used for the production of a variety of industrially important products, such as butyl rubber. Both of propylene and isobutylene are obtained through the catalytic or steam cracking of fossil feedstocks, and the development of a commercially viable process for the direct conversion of alcohol to either material would accordingly be of great interest as fossil resources are depleted and/or become more costly to use, especially in consideration of increased demand for both of propylene and isobutylene.

Natural raw materials are in particular substances which are obtained by processing from plants or parts of plants (or even animals). Characteristic of raw materials from renewable sources is a significantly high proportion of the carbon isotope ¹⁴ C. By its determination, the proportion of renewable raw materials can be determined experimentally. Renewable resources differ from those obtained through chemical synthesis or petroleum processing in that they are less homogeneous. The composition of the renewable resources varies considerably as the composition of natural resources depend on factors such as climate and region in which the plant grows, season of harvest, variations between biological species and subspecies and the type of extraction process used in extraction (extrusion, centrifugation, filtration, distillation, cutting, pressing, etc.).

Fusel oils are formed as a by-product of alcoholic fermentation and consist of a mixture of several alcohols comprised mainly of amyl alcohols along with lesser amounts of propanol, n-butanol, and iso-butanol depending upon the purification process employed. Depending on the carbohydrate source for the fermentation process, and the organism used, fusel oil levels are typically between 0.2-3.0% as a relative percent of the target alcohol produced. Fusel oils are co-products of alcohol fermentation. These oils, which are produced by yeast in anaerobiosis from nitrogenous materials, are recovered after rectification or on the middle plates of a column.

Fusel oils, occasionally referred to as “amyl oils” or “fusels”, have compositions which vary depending on their origin (potato, beet, wheat, barley, etc.). They are a mixtures of 5% to 20% of water, 60% to 95% of alcohols mainly consisting of linear or branched alkanols containing from 2 to 5 carbon atoms, of impurities (furfurols, ethers, fatty acids, etc.) which, in extreme cases, may be up to 15% Ethanol, 2-methyl 1-butanol and 3-methyl 1-butanol. Ethanol, 2-methyl 1-butanol and 3-methyl 1-butanol could be used as starting material for the preparation of several olefins which find application in various industries.

Some of the olefins (like Isobutylene, isoamylene) are most commonly used as a starting material for other products as opposed to being used as is for some final applications. While not an exhaustive one, the literature reveals several uses of olefins isobutylene and/or isoamylene. These include (i) hydrocarbon resin modification (softening point/Tg/molecular weight control), (ii) fuel additives via oligomerization (typically dimerization) for octane boosters or via etherification with methanol or ethanol, (iii) synthetic building block such as precursor to diolefins, flavor/fragrance enhancers, antioxidants, typically alkyl phenols, or as synthon for fine chemicals or pharmaceutical ingredients preparation.

Thus, an object of the present invention, was to produce olefins from alcohols which are found in renewable feedstock.

The further object of the present invention was to produce olefins having pMC greater than 90 using renewable raw materials which are comparable in quality and yield when produced using pure raw materials.

Yet another object of the present invention, was to develop a robust process which would yield olefins of high quality, higher yield and having pMC greater than 90 irrespective of the nature of the raw materials used (renewable source with several impurities or pure source).

### SUMMARY OF THE INVENTION

Surprisingly the above objects have been achieved by the process of the present invention.

Thus, the first aspect of the present invention is directed to a process for preparing a lower olefin with 4 to 7 carbon atoms, comprising the steps of:
a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt.-%.
b) oxidation of the primary alcohol to the corresponding carboxylic acid, and
c) oxidative decarboxylation of the carboxylic acid to an olefin having one carbon atom less than the starting alcohol.

The second aspect of the present invention is directed to a process where the starting material namely the C₅-C₈ primary alcohol has a pMC greater than 90 when measured by a method as described in the ASTM norm D6866 (the current version is D6866-22), which defines the concept of “percent Modern Carbon” or pMC. A “bio-based” material has a pMC of 100%.

The third aspect of the present invention is directed to a process, wherein the starting material namely the linear and/or branched C₅-C₈ primary alcohol which has at least one hydrogen atom attached to the carbon atom adjacent to the one bearing the alcohol group, at a concentration of 60-99 wt. % is converted to at least one lower hydrocarbon having C₄-C₇ carbon atoms at a yield of at least about 80 wt. % of the maximum theoretical molar yield.

Another aspect of the present invention is directed to a process for preparing isobutene starting from 3-methyl-1-butanol obtained from fusel oils using the combination of oxidation of the alcohol to 3-methylbutanoic acid and oxidative decarboxylation to isobutene. Although the individual steps are known reaction, they have not been described for this particular substrate, and they have not been combined as a means of obtaining isobutene with a pMC greater than 90 (pMC>90%).

### DESCRIPTION OF THE INVENTION

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are inter-changeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, min, hours, days, weeks, months or even years between such steps, unless otherwise indicated in the application as set forth herein above or below.

Furthermore, the ranges defined throughout the specification include the end values as well, i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment but may refer to the same embodiment. Further, as used in the following, the terms “preferably”, “more preferably”, “even more preferably”, “most preferably” and “in particular” or similar terms are used in conjunction with optional features, without restricting alternative possibilities. Thus, features introduced by these terms are optional features and are not intended to restrict the scope of the claims in any way.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any one of the claimed embodiments can be used in any combination.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

The term “yield” is defined as the amount of product obtained per unit weight of raw material and may be expressed as g product /g substrate. Yield may be expressed as a percentage of the theoretical yield. “Theoretical yield” is defined as the maximum amount of product that can be generated per a given amount of substrate as dictated by the stoichiometry of the metabolic pathway used to make the product. For example, if the theoretical yield for one typical conversion of glucose to isobutanol is 0.41 g/g, the yield of butanol from glucose of 0.39 g/g would be expressed as 95% of theoretical or 95% theoretical yield.

The term “biofuel precursor” refers to an organic molecule in which all of the carbon contained within the molecule is derived from biomass and is thermochemically or biochemically converted from a feedstock into the precursor. A biofuel precursor may be a biofuel in its own right or may be configured for conversion, either chemically or biochemically, into a biofuel with different properties. Biofuel precursors include, but are not limited to, 1-propanol, 2-propanol, 1-butanol, 2-butanol, isobutanol, 1-pentanol, isopentanol (3-methyl-1-butanol), 3-pentanol, 2-methyl-1-butanol, or neopentanol.

The terms “alkene” and “olefin” are used interchangeably herein to refer to non-aromatic hydrocarbons having at least one carbon-carbon double bond.

“Carbon of atmospheric origin” as used herein refers to carbon atoms from carbon dioxide molecules that have recently (e.g., in the last few decades) been free in the earth's atmosphere. Such carbon atoms are identifiable by the ratio of particular radioisotopes as described herein. “Green carbon”, “atmospheric carbon”, “environmentally friendly carbon”, “life-cycle carbon”, “non-fossil fuel based carbon”, “non-petroleum based carbon”, “carbon of atmospheric origin”, and “biobased carbon” are used synonymously herein.

“Carbon of fossil origin” as used herein refers to carbon of petrochemical origin. Carbon of fossil origin is identifiable by means described herein. “Fossil fuel carbon”, “fossil carbon”, “polluting carbon”, “petrochemical carbon”, “petrocarbon” and “carbon of fossil origin” are used synonymously herein.

The term “ASTM” refers to the American Society of Testing and Materials, which defines testing procedures and specifications for all petroleum products manufactured and sold commercially.

Fusel oil as used herein refers to the products that are formed as a by-product of alcoholic fermentation, and consist of a mixture of several alcohols comprised mainly of amyl alcohols along with lesser amounts of propanol, n-butanol, and isobutanol depending upon the purification process employed.

“Renewably-based” or “renewable” denote that the carbon content of the biofuel precursor and subsequent products is from a “new carbon” source as measured by ASTM test method D 6866-05, “Determining the Biobased Content of Natural Range Materials Using Radiocarbon and Isotope Ratio Mass Spectrometry Analysis”, incorporated herein by reference in its entirety.

### Process

The present invention is directed to a process for preparing a lower olefin with 4 to 7 carbon atoms, comprising the steps of:
a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt. %,
b) oxidation of the primary alcohol to the corresponding carboxylic acid, and
c) oxidative decarboxylation of the carboxylic acid to an olefin having one carbon atom less than the starting alcohol.

### Step a)

In step (a), the linear and/or branched C₅-C₈ primary alcohol has at least one hydrogen atom attached to the carbon atom adjacent to the one bearing the alcohol group.

The linear and/or branched C₅-C₈ primary alcohol has at least one hydrogen atom attached to the carbon atom adjacent to the one bearing the alcohol group that is the alpha carbon atom.

In an embodiment, the linear and/or branched C₅-C₈ primary alcohol has at least one hydrogen atom attached to the carbon atom adjacent to the one bearing the alcohol group that is the alpha carbon atom and the beta carbon atom (i.e the carbon atom attached to the first carbon atom adjacent to the one bearing the alcohol group) also has at least one hydrogen atom.

In a further embodiment, the C₅-C₈ primary alcohol is obtained from natural sources or obtained from fermentation process.

In an embodiment the C₅-C₈ primary alcohol is obtained from fusel oil.

### Fusel Oil

Fusel oil is well known in the art and comprises a mixture of light alcohols, fatty esters, terpenes and furfural. The alcohols comprised in fusel oil are mainly propanol, butanol, amyl alcohol, isoamyl alcohols and hexanol and optionally heavier linear alcohols such as C₇ or C₈ alcohols. Fusel oils, occasionally referred to as “amyl oils” or “fusels”, have compositions which vary depending on their origin (potato, beet, wheat, barley, etc. musts).

Fusel oils form colourless or yellowish liquids, which have a characteristic odour. They have a density of about 0.83. Their boiling point is far from constant, since they are complex mixtures of substances with a very variable boiling point. Boiling commences at about 80° C. and rises to 130-134° C. They are insoluble in water and are usually washed with water and separated out by settling of the phases in order to reduce the amount of ethanol they contain by about 4% to 5%. It should be noted that fusel alcohols are natural alcohols directly produced via biotechnology in distilleries, without any intermediate chemical step.

Fusel oil may be obtained by several processes well known from the skilled person, e.g. by direct removal in the distillation column and cooling. The removed fraction can be purified e.g. by extraction and decantation. A liquid/liquid extraction by addition of water followed by a decantation leads to the formation of two phases. The upper phase comprises mainly amyl and butyl alcohols, slightly soluble in water. The various fractions of fusel oil may also be separated by using adsorbents, which are regenerated thereafter. Among the tested adsorbents, granulated vegetal activated charcoal is preferred since it is able to adsorb eight times its weight of fusel oil.

In an embodiment the fusel oil contains a mixture of linear or branched C₅ alcohols, C₄ alcohols or C₃ alcohols.

According to a preferred embodiment, C₅ branched alcohol present in the initial composition is a mixture of isoamyl alcohol and amyl alcohol, i.e. 3-methylbutan-1-ol (isoamyl alcohol) and 2-methylbutan-1-ol (amyl alcohol).

According to a preferred embodiment, the initial composition comprises at least 30 wt. %, preferably at least 40 wt. %, more preferably at least 50 wt. %, more preferably at least 60 wt. %, even more preferably at least 70 wt. % C₅ branched alcohols, based on the total weight of the composition.

C₄ alcohols may also be present in the initial composition, for example, butan-1-ol and 2-methylpropan-1-ol. The initial composition may comprise one of these C₄ alcohols or both.

C₃ alcohols may also be present in the initial composition, for example, n-propanol. The initial composition may comprise 0.01 to 20 wt. % of C₃ alcohol.

Fusel oil may further contain hexanol and optionally heavier linear alcohols such as C₇ or C₈ alcohols.

Fusel oil is a mixture of 5% to 20% of water, 60% to 95% of alcohols mainly consisting of linear or branched alkanols containing from 2 to 5 carbon atoms of impurities including but not limiting to furfurals, ethers, fatty acids, etc. which, may be up to 15%.

In an embodiment the composition of fusel oil is as follows:
Ethanol 5 to 40%,
1-Propanol 1 to 8%,
2-Propanol 0 to 1%,
2-Methylpropanol 5 to 15%,
1-Butanol-0 to 1%,
2-Methyl 1-butanol 10 to 30%,
3-Methyl 1-butanol (isoamyl alcohol) 25 to 70%,
   the combination of alkanols representing 100%.

In an embodiment, the C₅-C₈ primary alcohol is selected from 2-methyl-1-butanol (active amyl alcohol), 3-Methyl 1-butanol(isoamyl alcohol, isopentanol), n-pentanol, n-hexanol, 2-methylpentanol, n-heptanol, n-octanol, 2-ethylhexanol or mixtures thereof.

In a preferred embodiment, the C₅-C₈ primary alcohol is 3-Methyl 1-butanol (isoamyl alcohol).

In an embodiment, the stream which is obtained from the fusel oil contains the C₅-C₈ primary alcohol at a concentration in the range of 60-99 wt % preferably in the range of 60-95 wt %, preferably in the range of 60-90 wt %, preferably in the range of 60-85 wt %, preferably in the range of 60-80 wt %, more preferably in the range of 65-80 wt %, even more preferably in the range of 70-80 wt %.

In an embodiment, the stream which is obtained from the fusel oil contains the C₅-C₈ primary alcohol has a pMC greater than 90 when measured by a method as described in the ASTM norm D6866 (the current version is D6866-22), which defines the concept of “percent Modern Carbon” or pMC. Preferably the pMC is greater than 91, preferably greater than 93, preferably greater than 95, preferably greater than 96, preferably greater than 97, more preferably greater than 98, even more preferably greater than 99.

In an embodiment, the verification that a feedstock was derived from renewable raw materials is possible according to ASTM D6866 via ¹⁴C for example. A feedstock shall be regarded as “derived from renewable raw materials” for the purposes of this invention when the carbon-14 (C-14) presence therein corresponds substantially (to within not more than 6%) to the ASTM D6866 content of C-14 in atmospheric CO₂.

The C-14 content of a material may be determined by determining the decays of C-14 in this material by liquid scintillation. Such raw materials shall preferably be regarded as derived from renewable raw materials when they have a C-14 content displaying a radioactive decay of not less than 1.5 dpm/gC (decays per minute per gram of carbon), preferably 2 dpm/gC, more preferably 2.5 dpm/gC and yet more preferably 5 dpm/gC.

“Renewably-based” or “renewable” denote that the carbon content of the biofuel precursor and subsequent products is from a “new carbon” source as measured by ASTM test method D 6866-05, “Determining the Biobased Content of Natural Range Materials Using Radiocarbon and Isotope Ratio Mass Spectrometry Analysis”, incorporated herein by reference in its entirety. This test method measures the ¹⁴C/¹²C isotope ratio in a sample and compares it to the ¹⁴C/¹²C isotope ratio in a standard 100% biobased material to give percent biobased content of the sample. “Biobased materials” are organic materials in which the carbon comes from recently (on a human time scale) fixated CO₂ present in the atmosphere using sunlight energy (photosynthesis). On land, this CO₂ is captured or fixated by plant life (e.g., agricultural crops or forestry materials). In the oceans, the CO₂ is captured or fixated by photosynthesizing bacteria or phytoplankton. For example, a biobased material has a ¹⁴C/¹²C isotope ratio greater than 0. Contrarily, a fossil-based material, has a ¹⁴C/¹²C isotope ratio of about 0. The term “renewable” with regard to compounds such as alcohols or hydrocarbons (linear or cyclic alkanes/alkenes/alkynes, aromatic, etc.) refers to compounds prepared from biomass using thermochemical methods (e.g., Fischer-Tropsch catalysts), biocatalysts (e.g., fermentation), or other processes, for example as described herein.

A small amount of the carbon atoms of the carbon dioxide in the atmosphere is the radioactive isotope ¹⁴C. This ¹⁴C carbon dioxide is created when atmospheric nitrogen is struck by a cosmic ray generated neutron, causing the nitrogen to lose a proton and form carbon of atomic mass 14 (¹⁴C), which is then immediately oxidized to carbon dioxide. A small but measurable fraction of atmospheric carbon is present in the faun of 14CO₂. Atmospheric carbon dioxide is processed by green plants to make organic molecules during the process known as photosynthesis. Virtually all forms of life on Earth depend on this green plant production of organic molecule to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from fossil fuels does not have the signature 14C:12C ratio of renewable organic molecules derived from atmospheric carbon dioxide. Furthermore, renewable organic molecules that biodegrade to CO₂ do not contribute to global warming as there is no net increase of carbon emitted to the atmosphere.

Assessment of the renewably based carbon content of a material can be performed through standard test methods, e.g. using radiocarbon and isotope ratio mass spectrometry analysis. ASTM International (formally known as the American Society for Testing and Materials) has established a standard method for assessing the biobased content of materials. The ASTM method is designated ASTM-D6866.

The application of ASTM-D6866 to derive “biobased content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modern carbon). If the material being analyzed is a mixture of present day radiocarbon and fossil carbon (containing very low levels of radiocarbon), then the pMC value obtained correlates directly to the amount of biomass material present in the sample.

The C₅-C₈ primary alcohols used in the present invention have pMC values of at least greater than 90, preferably pMC values of at least greater than 95, preferably pMC values of at least greater than 98, more preferably pMC values of at least greater than 99, more preferably pMC values of at least about 100, inclusive of all values and subranges there-between.

### Step b)

In step b), the linear and/or branched C₅-C₈ primary alcohol which has at least one hydrogen atom attached to the carbon atom adjacent to the one bearing the alcohol group is converted to the corresponding carboxylic acid.

The linear and/or branched C₅-C₈ primary alcohol has at least one hydrogen atom attached to the carbon atom adjacent to the one bearing the alcohol group that is the alpha carbon atom.

In an embodiment, the linear and/or branched C₅-C₈ primary alcohol has at least one hydrogen atom attached to the carbon atom adjacent to the one bearing the alcohol group that is the alpha carbon atom and the beta carbon atom (i.e the carbon atom attached to the first carbon atom adjacent to the one bearing the alcohol group) also has at least one hydrogen atom.

In an embodiment, the oxidation of the alcohol is carried out using oxidizing agents selected from oxygen, hydrogen peroxide or nitric acid.

Preferably the oxidizing agent used in this step are oxygen or nitric acid. More preferably the oxidizing agent used is nitric acid.

### Oxidation Using Nitric Acid

In an embodiment, the concentration of nitric acid used is in the range of 30 wt. %-65 wt. %, preferably in the range of 50 wt. %-65 wt. %, more preferably in the range of 60 wt. %-65 wt. %.

In an embodiment, the weight ratio/molar ratio of the oxidant i.e Nitric acid to that of the primary alcohol is in the range of 10:1, preferably 8:1, more preferably 6:1, even more preferably 5:1.

In an embodiment, the reaction is carried out at a temperature of less than 40° C., preferably the temperature is less than 35° C., more preferably the temperature is less than 30° C.

In an embodiment, the reaction is carried out at 35° C. when fusel oil is used as a source of alcohol.

In an embodiment, the reaction is carried out at 0° C., when pure alcohol is used as a reactant.

In an embodiment, the reaction is carried out in the presence of a solvent.

In an embodiment, the solvent is selected from aliphatic hydrocarbons, such as hexane, heptane, octane, nonane, decane and also petroleum ether, or halogenated hydrocarbons such as bromopropane, methylene chloride or dichloromethane, chloroform, tetrachloroethylene, aromatic hydrocarbons, such as benzene, toluene, the xylenes and mesity-lene, aliphatic C3-C8-ethers, such as 1,2-dimethoxyethane (DME), diethylene glycol dimethyl ether (diglyme), diethyl ether, dipropyl ether, methyl isobutyl ether, tert-butyl methyl ether and tert-butyl ethyl ether, dimethoxymethane, diethoxymethane, dimethylene glycol dimethyl ether, dimethylene glycol diethyl ether, trimethylene glycol dimethyl ether, trimethylene glycol diethyl ether, tetramethylene gly-col dimethyl ether, cycloaliphatic hydrocarbons, such as cyclohexane and cycloheptane, alicyclic C3-C6 ethers, such as tetrahydrofuran (THF), tetrahydropyran, 2-methyltetra-hydrofuran, 3-methyltetrahydrofuran, 1,3-dioxolane, and 1,4-dioxane, 1,3,5-trioxane, short-chain ketones, such as acetone, ethyl methyl ketone and isobutyl methyl ketone.

In a preferred embodiment, the solvent is dichloromethane.

In an embodiment, the oxidation of the alcohol is performed in the presence of a nitric acid, wherein the reaction is carried out for a time period in the range of 60 min to 240 min, preferably in the range of 60 min to 180 min, more preferably in the range of 60 min to 100 min.

In an embodiment, the carboxylic acid formed was isolated by distillation.

### Oxidation in the Presence of O₂

In an embodiment, the oxidation reaction is carried out in the presence of a gas stream containing O₂ as the oxidizing agent and a heterogeneous catalyst comprising metal catalyst.

In an embodiment, the oxidation reaction is carried out in the presence of a gas stream containing O₂ as the oxidizing agent. Oxygen can be used undiluted or diluted. The oxygen can be diluted with other inert gases like N₂, Ar or CO₂, e.g in the form of air.

In a preferred embodiment of the invention oxygen is used undiluted.

In an embodiment, the oxidation reaction is carried out in the presence of an O₂ stream which has a flow rate of 1-10 lit/hour, preferably, 2-8 lit/hour, more preferably 3-7 lit/hour, more preferably 4-6 lit/hour.

In a preferred embodiment, the oxidation reaction is carried out in the presence of an O₂ stream which has a flow rate of 5 lit/hour.

The process according to the invention is carried out in the presence of a catalyst. The catalyst comprises at least one catalytically active metal. In the process according to the invention the catalytically active metal can be selected from the elements selected from the groups 8, 9, 10 and 11 of the periodic table (according to IUPAC nomenclature). The elements of group 8, 9, 10 and 11 of the periodic table comprise iron, cobalt, nickel, ruthenium, rhodium, palladium, osmium, iridium, platinum, copper, silver and gold.

In a preferred embodiment, the catalytically active metal is selected from elements from the groups 10 and 11 of the periodic table (according to IUPAC nomenclature).

In a preferred embodiment, the catalytically active metal is selected from elements selected from the group consisting of platinum, palladium and gold or mixtures thereof.

In a preferred embodiment of the invention the catalytically active metal is platinum.

The catalytically active metal can be used in any form, e.g. unsupported or on a support. The catalytically active metal can be used in an unsupported form, for example as a powder, a mesh, a sponge, a foam or a net. In a preferred embodiment, the catalytically active metal is on a support.

In an embodiment, the metal catalyst is on a support selected from active carbon, silica or alumina. Preferably the support is selected from active carbon.

The catalyst can optionally comprise one or more so called promotors, which enhance the activity of the catalytically active metal. Examples for such promotors are bismuth (Bi), antimony (Sb), lead (Pb), cadmium (Cd), tin (Sn), tellurium (Te), cerium (Ce), selenium (Se) or thallium (Tl).

In a preferred embodiment, the catalyst comprises at least one promotor selected from the group consisting of bismuth (Bi), antimony (Sb), lead (Pb), cadmium (Cd), tin (Sn) and tellurium (Te). In a preferred embodiment, the catalyst comprises at least one promotor selected from the group consisting of bismuth (Bi), lead (Pb) and cadmium (Cd).

The promotors can for example be employed as metals, nitrates, acetates, sulphates, citrates, oxides, hydroxides or chlorides and mixtures thereof.

In case a promotor is employed, suitable molar ratios of the catalytically active metal and the promotor are in the range from 1:0.01 to 1:10, preferably 1:0.5 to 1:5, more preferably from 1:0.1 to 1:3.

The promotors can for example be present on the support or can be added separately to the process.

The term “on a support” encompasses that the catalytically active metal and/or promotor can be located on the outer surface of a support and/or on the inner surface of a support. In most cases, the catalytically active metal and/or promotor will be located on the outer surface of a support and on the inner surface of a support.

In case the catalytically active metal is on a support, the catalyst comprises the catalytically active metal, the support and optionally promotors.

In an embodiment, the reaction is carried out at a temperature in the range of 70° C. to 100° C., preferably in the range of 80° C. to 100° C., preferably in the range of 80° C. to 95° C., preferably in the range of 80° C. to 90° C., preferably in the range of 80° C. to 85° C., more preferably the temperature is about 80° C.

In an embodiment, the oxidation of the alcohol is performed in the presence of a gas stream of O₂, wherein the reaction is carried out for a time period of 120 min to 24 hours, preferably in the range of 120 min to 12 hours, more preferably in the range of 120 min to 8 hours, even more preferably in the range of 120 min to 240 min.

### Step c)

In an embodiment in step c), the oxidative decarboxylation of the carboxylic acid to a lower olefin having C₄-C₇ carbon atoms is carried out.

In an embodiment the oxidative decarboxylation is carried out in the presence of a homogenous catalyst wherein the homogenous catalyst comprises at least one metal or its salt or complex and a ligand.

In an embodiment, the at least one metal is selected from Nickel, Palladium, or Platinum. Preferably the metal is Palladium.

In an embodiment, the at least one metal or its salt or complex is selected from PdCl₂, tetrakis(triphenylphosphine) palladium, dichlorobis(triphenylphosphine) palladium, tris(dibenzylideneacetone) dipalladium [Pd2(dba)3], bis(dibenzylideneacetone) dipalladium [Pd(dba)2], palladium acetate, dichloro(1,5-cyclooctadiene) palladium and bis[cinnamyl palladium(II)]chloride.

Preferably the metal salt is PdCl₂.

In an embodiment, the homogeneous catalyst includes a ligand selected from the group consisting of 5-(di-tert-butylphosphino)-1′,3′,5′-triphenyl-1′H-1,4′-bipyrazole, bis(2-methyl-2-propanyl)(2′,4′,6′-triisopropyl-3,6-dimethoxy-2-biphenylyl)phosphine, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyldi-1-adamantyl phosphine, (5-diphenylphosphanyl-9,9-dimethylxanthen-4-yl)-diphenylphosphane, (R)-1-[(SP)-2-(diphenylphosphino)ferrocenyl]ethyldicyclohexyl phosphine, dicyclohexyl-[2-[2,6-di(propan-2-yloxy)phenyl]phenyl]phosphane, bis[5-(di(1-adamantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino) ethane, 4,5-bis(diphenylphosphino)-9,9-dimethyl-xanthene (xant-phos), 1,1′-bis(diphenylphosphino)ferrocene (dppf), bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane and 1,1′-bis(diphenylphosphino) ferrocene (dppf), Triphenyl phosphine (PPh3), Xantphos ((9,9-Dimethyl-9H-xanthene-4,5-diyl)bis(diphenylphosphane)), SPANphos (4,4,4′,4′,6,6′-Hexamethyl-3,3′,4,4′-tetrahydro-2,2′-spirobi[[1]benzopyran]-8,8′-diyl)bis(diphenylphosphane)), 2,2′-Bis-(diphenylphosphino)-benzophenone.

Preferably the ligand is selected from bis(2-(diphenyl-phosphino)phenyl)ether [DPE-phos], Triphenyl phosphine (PPh3), Xantphos ((9,9-Dimethyl-9H-xanthene-4,5-diyl)bis(diphenylphosphane)), SPANphos (4,4,4′,4′,6,6′-Hexamethyl-3,3′,4,4′-tetrahydro-2,2′-spirobi[[1]benzopyran]-8,8′-diyl)bis(diphenylphosphane)), 2,2′-Bis-(diphenylphosphino)-benzophenone.

More preferably the ligand is bis(2-(diphenyl-phosphino)phenyl)ether [DPE-phos].

In an embodiment, the homogenous catalyst is used at a concentration of less than 5 mol. % Preferably less than 3 mol %, more preferably less than 2 mol %.

In an embodiment, the catalyst is used at a concentration in the range of 0.1 mol. % to 2 mol. % based on the carboxylic acid in step (b).

In an embodiment, the mole ratio of the ligand to the metal catalyst or its salt or complex is in the range of 5:1 to 1:1, preferably the ratio of the ligand to the metal catalyst metal catalyst or its salt or complex is 2:1.

In an embodiment, step c) is carried out in the presence of an acid anhydride.

In an embodiment, the decarboxylation of the acid to a lower olefin having C₄-C₇ carbon atoms in step c) is carried out in the presence of a carboxylic anhydride different from the coupling product of the carboxylic acids obtained in step b).

In another embodiment, the acid anhydride is selected from acetic anhydride, propanoic anhydride, butanoic anhydride, or maleic anhydride. Preferably the acid anhydride is acetic anhydride.

In an embodiment, step (c) is carried out at a reaction temperature of less than 150° C., preferably less than 145° C., preferably in the range of 130° C. to 150° C., more preferably at a temperature in the range of 130° C. to 145° C.

In a further embodiment, step c) is carried out for a time period in the range of 60 min to 300 min, preferably 100 min to 300 min, more preferably in the range of 120 min to 240 min, more preferably in in the range of 60 to 240 minutes.

In an embodiment, the olefin having C₄-C₇ carbon atoms is selected from is 1-butene, isobutylene, 1-pentene, isopentene, 1-hexene, 2-hexene, 3-hexene, 4-methyl-1-pentene, Preferably the olefin is isobutylene.

In an embodiment of the present invention, the process comprises the steps of:
a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt.-%.
b) oxidation of the primary alcohol to the corresponding carboxylic acid, and
c) oxidative decarboxylation of the carboxylic acid to an olefin having one carbon atom less than the starting alcohol,
   wherein linear or branched C₅-C₈ alcohol at a concentration of 60-99 wt. % is converted to at least one lower hydrocarbon having C₄-C₇ carbon atoms at a yield of at least about 80 wt. %. of the maximum theoretical molar yield.

In an embodiment of the present invention, the process comprises the steps of:
a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt. %,
b) oxidation of the primary alcohol to the corresponding carboxylic acid in the presence of oxidizing agents selected from nitric acid or O₂, and the oxidation of the alcohol is performed in the presence heterogeneous material comprising platinum, and
c) oxidative decarboxylation of the carboxylic acid to an olefin having one carbon atom less than the starting alcohol, wherein the oxidative decarboxylation is carried out in the presence of a homogenous catalyst at a concentration of less than 5 mol. % preferably less than 3 mol %, more preferably less than 2 mol %.
   wherein linear or branched C₅-C₈ alcohol which is isoamyl alcohol at a concentration of 60-99 wt. % is converted to at least one lower hydrocarbon having C₄-C₇ carbon atom which is isobutylene, at a yield of at least about 80 wt. % of the maximum theoretical molar yield.

### Advantages of the Present Invention

1. The process according to the invention enables the preparation of olefins with high yield and high selectivity under mild conditions, both of temperature and pressure, while requiring only moderate to low amounts of catalyst.
2. The process can be conducted with no or low amounts of organic solvent, thus avoiding or minimizing environmentally problematic waste stream.
3. A further advantage of the process of the invention is that the desired olefin is obtained in a high concentration in the reaction mixture, thus minimizing down-stream isolation steps.
4. The process of the present invention enables the formation of olefins in comparable yield and purity irrespective of the source of the primary alcohol used as the starting material. (Pure primary alcohol or alcohol isolated from fusel oil). Thus, the impurities of the starting material do not hamper the yield and purity of the final product (olefin in this case).
5. The process according to the present invention uses readily available reagents and thereby reduces the overall cost of the process, thereby making it industrially viable process which can be easily scaled up.
6. The process according to the present invention, uses 3-methyl-1-butanol from fusel oil as the starting material and using the combination of oxidation of the alcohol to 3-methylbutanoic acid and oxidative decarboxylation to isobutene. Although the individual steps are known reaction, they have not been described for this particular substrate, and they have not been combined as a means of obtaining olefin (isobutene) with a pMC>90% and thus can be considered to be inventive.
7. The olefin (isobutene) that is obtained would also have a pMC value greater than 90 which can be deduced from the fact that the input stream comprising linear or branched C₅-C₈ primary alcohol has a pMC value greater than 90 (which is obtained in natural sources—namely the fusel oil).

### Embodiments

In the following, there is provided a list of embodiments to further illustrate the present disclosure without intending to limit the disclosure to the specific embodiments listed below.
1. A process for preparing a lower olefin with 4 to 7 carbon atoms, comprising the steps of:
   a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt.-%.
   b) oxidation of the primary alcohol to the corresponding carboxylic acid, and
   c) oxidative decarboxylation of the carboxylic acid to an olefin having one carbon atom less than the starting alcohol.
2. The process according to embodiment 1, wherein the stream comprising linear and/or branched C₅-C₈ primary alcohol which has at least one hydrogen atom attached to the carbon atom adjacent to the one bearing the alcohol group has a pMC at least greater than 90, preferably pMC values of at least greater than 95, preferably pMC values of at least greater than 98, more preferably pMC values of at least greater than 99, more preferably pMC values of at least about 100, inclusive of all values and subranges there-between, when measured by a method as described in the ASTM norm D6866.
3. The process according to embodiment 2, wherein the stream comprising linear or branched C₅-C₈ primary alcohol which has at least one hydrogen atom attached to the carbon atom adjacent to the one bearing the alcohol group is obtained from natural sources.
4. The process according to embodiment 2, wherein the stream comprising linear or branched C₅-C₈ primary alcohol which has at least one hydrogen atom attached to the carbon atom adjacent to the one bearing the alcohol group is obtained from fermentation process.
5. The process according to embodiment 1, wherein the stream comprising linear and/or branched C₅-C₈ primary alcohol has at least one hydrogen atom attached to the carbon atom adjacent to the one bearing the alcohol group that is the alpha carbon atom.
6. The process according to embodiment 5, wherein the stream comprising the linear and/or branched C₅-C₈ primary alcohol has at least one hydrogen atom attached to the carbon atom adjacent to the one bearing the alcohol group that is the alpha carbon atom and the beta carbon atom (i.e the carbon atom attached to the first carbon atom adjacent to the one bearing the alcohol group) also has at least one hydrogen atom.
7. The process according to any of the embodiments 1 to 6, wherein the linear or branched C₅-C₈ primary alcohol which has at least one hydrogen atom attached to the carbon atom adjacent to the one bearing the alcohol group is selected from 2-methyl-1-butanol (active amyl alcohol), 3-methylbutan-1-ol (isoamyl alcohol, (isopentanol), n-pentanol, n-hexanol, 2-methylpentanol, n-heptanol, n-octanol, 2-ethylhexanol.
8. The process according to any of the embodiments 1 to 7, wherein the linear or branched C₅-C₈ primary alcohol which has at least one hydrogen atom attached to the carbon atom adjacent to the one bearing the alcohol group is 3-methylbutan-1-ol (isoamyl alcohol, isopentanol).
9. The process according to any of the embodiments 1 to 8, wherein the stream comprises linear or branched C₅-C₈ primary alcohol which has at least one hydrogen atom attached to the carbon atom adjacent to the one bearing the alcohol group at a concentration in the range of 60 wt. %-99 wt. %, preferably in the range of 60 wt. %-95 wt. %, preferably in the range of 60 wt. %-90 wt. %, preferably in the range of 60 wt. %-85 wt. %, preferably in the range of 60 wt. %-80 wt. %, more preferably in the range of 65 wt. %-80 wt. %, even more preferably in the range of 70 wt. %-80 wt. %.
10. The process according to embodiment 1, wherein in step (b), the oxidation of the primary alcohol is performed using an oxidizing agent selected from O₂, H₂O₂ or nitric acid.
11. The process according to embodiment 10, wherein in step (b), the oxidation of the primary alcohol is performed using nitric acid.
12. The process according to embodiment 11, wherein in step (b), the oxidation of primary alcohol is performed using nitric acid with a concentration in the range of 30 wt. %-65 wt. %, preferably in the range of 50 wt. %-65 wt. %, more preferably in the range of 60 wt. %-65 wt. %.
13. The process according any of the embodiments 10 to 11, wherein in step (b), the oxidation of the primary alcohol is performed at a reaction temperature of less than 40° C., preferably the temperature is less than 35° C., more preferably the temperature is less than 30° C.
14. The process according any of the embodiments 10 to 11, wherein in step (b), the oxidation of the primary alcohol is performed in the presence of Nitric acid wherein the reaction is carried out for a time period in the range of 60 min to 240 min, preferably in the range of 60 min to 180 min, more preferably in the range of 60 min to 100 min.
15. The process according to embodiment 10, wherein in step (b), the oxidation of the primary alcohol is performed in the presence of a gas stream containing O₂ as the oxidizing agent which has a flow rate of 1-10 lit/hour, preferably, 2-8 lit/hour, more preferably 3-7 lit/hour, more preferably 4-6 lit/hour and a heterogeneous material comprising metal catalyst selected from the group selected from platinum, palladium, gold or mixtures thereof.
16. The process according to embodiment 15, wherein in step (b), the oxidation of the alcohol is performed in the presence heterogeneous material comprising platinum.
17. The process according to embodiments 15 to 16, wherein the metal catalyst is carried on inert support, wherein the support is selected from active carbon, silica or alumina which are optionally doped with bismuth cadmium or lead.
18. The process according to any of the embodiments 15 to 17, wherein the oxidation of the alcohol is performed at a reaction temperature of 80° C. to 100° C., preferably in the range of 80° C. to 95° C., preferably in the range of 80° C. to 90° C., preferably in the range of 80° C. to 85° C., more preferably the temperature is about 80° C.
19. The process according to any of the embodiments 15 to 17, wherein the oxidation of the primary alcohol is performed in the presence of a gas stream of O₂, wherein the reaction is carried out for a time period in the range of 120 min to 24 hours, preferably in the range of 120 min to 12 hours, more preferably in the range of 120 min to 8 hours, even more preferably in the range of 120 min to 240 min.
20. The process according to embodiment 1, wherein in step (c) oxidative decarboxylation of the acid to a lower olefin having C₄-C₇ carbon atoms is carried out in the presence of a homogenous catalyst.
21. The process according to embodiment 20, the homogenous catalyst comprises at least one metal or its salt or complex, and a ligand.
22. The process according to embodiment 21, wherein the at least one metal is selected from Nickel, Palladium or Platinum.
23. The process according to embodiment 22, wherein the at least one metal is Palladium.
24. The process according to any of the embodiments 21 to 23, wherein the at least one metal or its salt or complex is selected from PdCl₂, tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium [Pd(dba)₂], palladium acetate, dichloro(1,5-cyclooctadiene) palladium and bis[cinnamyl palladium(II)]chloride.
25. The process according to embodiment 21, wherein the homogeneous catalyst includes a ligand selected from the group consisting of 5-(di-tert-butylphosphino)-1′,3′,5′-triphenyl-1′H-1,4′-bipyrazole, bis(2-methyl-2-propanyl)(2′,4′,6′-triisopropyl-3,6-dimethoxy-2-biphenylyl)phosphine, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyldi-1-adamantyl phosphine, (5-diphenylphosphanyl-9,9-dimethylxanthen-4-yl)-diphenylphosphane, (R)-1-[(SP)-2-(diphenylphosphino)ferrocenyl]ethyldicyclohexyl phosphine, dicyclohexyl-[2-[2,6-di(propan-2-yloxy)phenyl]phenyl]phosphane, bis[5-(di(1-adamantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino) ethane, 4,5-bis(diphenylphosphino)-9,9-dimethyl-xanthene (xant-phos), 1,1′-bis(diphenylphosphino)ferrocene (dppf), bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane and 1,1′-bis(diphenylphosphino) ferrocene (dppf), Triphenyl phosphine (PPh3), Xantphos ((9,9-Dimethyl-9H-xanthene-4,5-diyl)bis(diphenylphosphane)), SPANphos (4,4,4′,4′,6,6′-Hexamethyl-3,3′,4,4′-tetrahydro-2,2′-spirobi[[1]benzopyran]-8,8′-diyl)bis(diphenylphosphane)), 2,2′-Bis-(diphenylphosphino)-benzophenone, preferably the ligand is selected from bis(2-(diphenyl-phosphino)phenyl)ether [DPE-phos], Triphenyl phosphine (PPh3), Xantphos ((9,9-Dimethyl-9H-xanthene-4,5-diyl)bis(diphenylphosphane)), SPANphos (4,4,4′,4′,6,6′-Hexamethyl-3,3′,4,4′-tetrahydro-2,2′-spirobi[[1]benzopyran]-8,8′-diyl)bis(diphenylphosphane)), 2,2′-Bis-(diphenylphosphino)-benzophenone, more preferably the ligand is bis(2-(diphenyl-phosphino)phenyl)ether [DPE-phos].
26. The process according to any of the embodiments 20 to 25, wherein in step (c), wherein the homogenous catalyst is used at a concentration of less than 5 mol. % preferably less than 3 mol %, more preferably less than 2 mol %.
27. The process according to any of the embodiments 20 to 25, wherein in step (c), the mole ratio of the ligand to the metal catalyst or its salt or its complex is 5:1 or 3:1; preferably the ratio is 2:1.
28. The process according to any of the embodiments 20 to 25, wherein in step (c), the decarboxylation of the acid to a lower hydrocarbon having C₄-C₇ carbon atoms is carried out in the presence of a carboxylic anhydride (acid anhydride) different from the coupling product of the carboxylic acids obtained in step b).
29. The process according to embodiment 26, wherein the acid anhydride is selected from acetic anhydride, propanoic anhydride, butanoic anhydride, or maleic anhydride.
30. The process according to embodiment 27, wherein the acid anhydride is acetic anhydride.
31. The process according to embodiments 20 to 29, wherein in step (c) decarboxylation of the acid to a lower hydrocarbon having C₄-C₇ carbon atoms is carried out at a reaction temperature of less than 150° C., preferably less than 145° C., preferably in the range of 130° C. to 150° C., more preferably at a temperature in the range of 130° C. to 145° C.
32. The process according to embodiments 20 to 30, wherein in step (c) decarboxylation of the acid to a lower hydrocarbon having C₄-C₇ carbon atoms is carried out for a time period in the range of 60 min to 300 min, preferably 100 min to 300 min, more preferably in the range of 120 min to 240 min, more preferably in in the range of 60 to 240 minutes.
33. The process according to embodiments 20 to 31, wherein the lower hydrocarbon having C₄-C₇ carbon atoms is selected from is 1-butene, isobutylene, 1-pentene, isopentene, 1-hexene, 2-hexene, 3-hexene, 4-methyl-1-pentene.
34. The process according to embodiment 33, wherein the lower hydrocarbon having C₄-C₇ carbon atoms is isobutylene.
35. The process according to any of the embodiments 1 to 33, wherein linear or branched C₅-C₈ primary alcohol which has at least one hydrogen atom attached to the carbon atom adjacent to the one bearing the alcohol group having a concentration of 60-99 wt % is converted to at least one lower hydrocarbon having C₄-C₇ carbon atoms at a yield of at least about 80 wt %. of the maximum theoretical molar yield,
36. The process according to any of the embodiments 1 to 35, wherein the process comprises the steps of:
   a) feeding into a reaction vessel a stream comprising linear or branched C₅-C₈ primary alcohol which has at least one hydrogen atom attached to the carbon atom adjacent to the one bearing the alcohol group, at a concentration of 60-99 wt.-%,
   b) oxidation of the primary alcohol to the corresponding carboxylic acid, and
   c) oxidative decarboxylation of the carboxylic acid to an olefin having one carbon atom less than the starting alcohol,
      wherein linear or branched C₅-C₈ primary alcohol at a concentration of 60-99 wt. % is converted to at least one lower hydrocarbon having C₄-C₇ carbon atoms at a yield of at least about 80 wt. % of the maximum theoretical molar yield.

### Examples

The invention is further illustrated by the following non-limiting examples.

### Materials

Crude/isolated from Fusel oil—Isoamyl alcohol was procured from Crop Energies AG.

The composition of the fusel oil used was as follows:

| | |
|---|---|
| Ethanol | 2.15% |
| Isobutanol | 13.38% |
| Butanol | 0.98% |
| Isoamyl alcohol | 77.23% |
| 2-Methylbutanol | 3.55% |
| Furfural | 0.21% |
| Hexanol | 0.21% |
| Isoamyl acetate | 0.13% |
| Benzaldehyde | 0.01% |
| Ethyl hexanoate | 0.09% |
| Phenyl ethanol | 0.20% |
| Phenyl ethyl acetate | 0.11% |
| Ethyl decanoate | 0.10% |
| Ethyl laurate | 0.05% |
| Hexanoic acid ethyl ester | 0.31% |
| Unknown impurities | 1.29% |

### Example 1: Oxidation of Pure Isoamyl Alcohol Using O₂

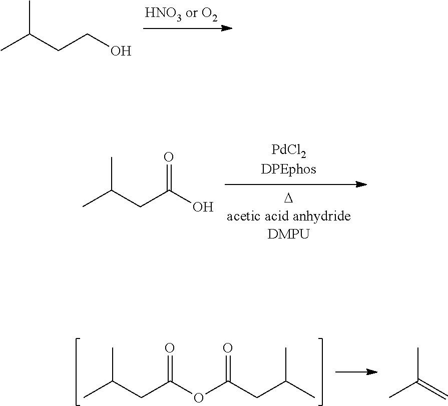

Isoamyl alcohol (10 g) was mixed with water (90 g). The catalyst Pt/C (3 g, 5 wt.-% Pt on C, 58 wt.-% water) was added, and the mixture was stirred at 80° C. for 24 h under a dioxygen atmosphere. After cooling down to ambient temperature, the mixture was diluted with ethyl acetate, filtered and the aqueous phase was extracted with ethyl acetate. The solvent was removed in vacuo. Yield: 85%

### Example 2: Oxidation of Pure Isoamyl Alcohol Using HNO₃

Nitric acid (55 g, 65%) was cooled down to 0° C. using an ice bath. While stirring under nitrogen, isoamyl alcohol (10 g) was added dropwise over 45 min. After complete addition, water (50 mL) was added, and the mixture was stirred for 10 min. The biphasic mixture was diluted with dichloromethane (100 mL) and stirred for 30 min. The phases were separated, and the organic phase was dried over sodium sulphate. The product isovaleric acid was isolated by distillation (190° C. oil bath, 120° C. top temperature). Yield: 94%

### Example 3: Oxidation of Fusel Oil (Containing 70% Isoamyl Alcohol) Using HNO₃

Nitric acid (57 g, 65%) was tempered to 35° C. in an oil batch. While stirring under nitrogen, fusel oil (14.5 g, containing 70% isoamyl alcohol) was added dropwise over 45 min. After complete addition, water (50 mL) was added, and the mixture was stirred for 10 min. The biphasic mixture was diluted with dichloromethane (100 mL) and water (100 mL) and stirred for 30 min. The phases were separated, and the organic phase was dried over sodium sulphate. The product isovaleric acid was isolated by distillation (200° C. oil bath, 170° C. top temperature). Yield: 84%

### Example 4: Decarboxylation of Isovaleric Acid to Isobutylene

Isovaleric acid (1.87 g) was mixed with PdCl₂ (1.76 mol %) and dis[(diphenylphosphino) phenyl]ether (DPE-Phos, 3.53 mol %) under nitrogen atmosphere. DMPU (N,N′-Dimethyl propylene urea, 36 g) was added and while stirring, acetic acid anhydride (2.53 g) was added slowly. After stirring for 15 min at ambient temperature, the reaction mixture was heated to 140° C. for 4 h. While heating, PdCl₂ dissolved completely which was indicated by a yellow coloration of the solution.

The end of the reaction was indicated by a red coloration of the reaction mixture. The formed isobutylene was collected using a gas collector for analytics and condensation in toluene. Yield: 85-87%

### Example 5: Oxidation of Pure 2-Methyl-1-Butanol Using HNO₃

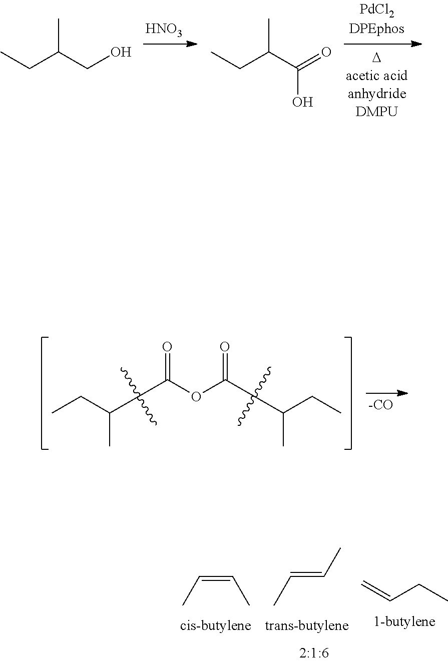

Nitric acid (5.5 g, 65%) was cooled down to 0° C. using an ice bath. While stirring under nitrogen, 2-methyl-1-butanol (1 g) was added dropwise over 15 min. After complete addition, water (50 mL) was added, and the mixture was stirred for 10 min. The biphasic mixture was diluted with dichloromethane (15 mL) and stirred for 30 min. The phases were separated, and the organic phase was dried over sodium sulphate. The product 2-methyl-1-butanoic acid was isolated by distillation (190° C. oil bath, 120° C. top temperature). Yield: 90%.

### Example 6: Decarboxylation of 2-Methyl-1-Butanoic Acid to Butylene Isomers

2-Methyl-1-butanoic acid (0.5 g) obtained in example 5, was mixed with PdCl₂ (1.76 mol %) and dis[(diphenylphosphino)phenyl]ether (DPE-Phos, 3.53 mol %) under nitrogen atmosphere. DMPU (N,N′-Dimethylpropyleneurea, 9.5 g) was added and acetic acid anhydride (0.68 g) was added slowly under stirring. After stirring for 15 min at ambient temperature, the reaction mixture was heated to 140° C. for 4 h. While heating, PdCl₂ dissolved complete, indicated by a yellow coloration of the solution.

The end of the reaction is indicated by a red coloration of the reaction mixture. The formed butylene isomers are collected using a gas collector for analytics and condensation in toluene. Yield: 81% containing 1-butylene, cis-butylene and trans-butylene in a ratio of 6:2:1.

TABLE 1

| | Acid %(product | Isobutylene % (product |
|---|---|---|
| Iso amyl alcohol source | of step b)) | of step c)) |
| Neat | 85-94 | 85 |
| Isolated from crude fusel | 84 | 85-87 |
| oil | | |

1.-20. (canceled)

21. A process for preparing a lower olefin with 4 to 7 carbon atoms, comprising the steps of:
a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt. %,
b) oxidation of the primary alcohol to the corresponding carboxylic acid, and
c) oxidative decarboxylation of the carboxylic acid to an olefin having one carbon atom less than the starting alcohol.

22. The process according to claim 21, wherein the stream comprising linear and/or branched C₅-C₈ primary alcohol which has at least one hydrogen atom attached to the carbon atom adjacent to the one bearing the alcohol group has a pMC greater than 90, when measured by a method as described in the ASTM norm D6866.

23. The process according to claim 22, wherein the stream comprising linear and/or branched C₅-C₈ primary alcohol which has at least one hydrogen atom attached to the carbon atom adjacent to the one bearing the alcohol group is obtained from natural sources or from a fermentation process.

24. The process according to claim 21, wherein the linear or branched C₅-C₈ primary alcohol which has at least one hydrogen atom attached to the carbon atom adjacent to the one bearing the alcohol group is selected from 2-methyl-1-butanol (active amyl alcohol), 3-methylbutan-1-ol (isoamyl alcohol, isopentanol), n-pentanol, n-hexanol, 2-methylpentanol, n-heptanol, n-octanol, or 2-ethylhexanol.

25. The process according to claim 21, wherein the linear or branched C₅-C₈ primary alcohol which has at least one hydrogen atom attached to the carbon atom adjacent to the one bearing the alcohol group is 3-methylbutan-1-ol (isoamyl alcohol, isopentanol).

26. The process according to claim 21, wherein in step (b), the oxidation of the primary alcohol is performed using an oxidizing agent selected from 02, H₂O₂ or nitric acid.

27. The process according to claim 26, wherein in step (b), the oxidation of the primary alcohol is performed using nitric acid at a concentration in the range of 40-65 wt. %.

28. The process according to claim 26, wherein in step (b), the oxidation of the primary alcohol is performed in the presence of a gas stream containing 02 as the oxidant and a heterogeneous catalyst comprising metal catalyst selected from platinum, palladium, gold or mixtures thereof.

29. The process according to claim 28, wherein in step (b), the oxidation of the primary alcohol is performed in the presence of platinum.

30. The process according to claim 21, wherein in step (c) oxidative decarboxylation of the acid to a lower olefin having C₄-C₇ carbon atoms is carried out in the presence of a homogenous catalyst.

31. The process according to claim 30, wherein the homogenous catalyst comprises at least one metal or its salt or complex, and a ligand.

32. The process according to claim 31, wherein the at least one metal is selected from nickel, palladium, or platinum.

33. The process according to claim 31, wherein the at least one metal or its salt or complex is selected from PdCl₂, tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium [Pd(dba)₂], palladium acetate, dichloro(1,5-cyclooctadiene) palladium, orbis[cinnamyl palladium(II)]chloride.

34. The process according to claim 31, wherein the homogeneous catalyst includes a ligand selected from 5-(di-tert-butylphosphino)-1′,3′,5′-triphenyl-1′H-1,4′-bipyrazole, bis(2-methyl-2-propanyl)(2′,4′,6′-triisopropyl-3,6-dimethoxy-2-biphenylyl)phosphine, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyldi-1-adamantyl phosphine, (5-diphenylphosphanyl-9,9-dimethylxanthen-4-yl)-diphenylphosphane, (R)-1-[(SP)-2-(diphenylphosphino)ferrocenyl]ethyldicyclohexyl phosphine, dicyclohexyl-[2-[2,6-di(propan-2-yloxy)phenyl]phenyl]phosphane, bis[5-(di(1-adamantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino) ethane, 4,5-bis(diphenylphosphino)-9,9-dimethyl-xanthene (xant-phos), 1,1′-bis(diphenylphosphino)ferrocene (dppf), bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane, or 1,1′-bis(diphenylphosphino) ferrocene (dppf).

35. The process according to claim 30, wherein in step (c), the decarboxylation of the acid to a lower olefin having C₄-C₇ carbon atoms is carried out in the presence of a carboxylic anhydride different from the coupling product of the carboxylic acids obtained in step b).

36. The process according to claim 35, wherein the carboxylic anhydride is selected from acetic anhydride, propanoic anhydride, butanoic anhydride, or maleic anhydride.

37. The process according to c claim 30, wherein the catalyst is used at a concentration in the range of 0.1 mol. % to 2 mol. % based on the carboxylic acid in step (b).

38. The process according to claim 21, wherein the lower olefin having C₄-C₇ carbon atoms is selected from is 1-butene, isobutylene, 1-pentene, 3-methyl-1 butene, hexene, 2-hexene, 3-hexene, or 4-methyl-1-pentene.

39. The process according to claim 21, wherein the stream comprising linear and/or branched C₅-C₈ primary alcohol which has at least one hydrogen atom attached to the carbon atom adjacent to the one bearing the alcohol group having a concentration of 60-99 wt. % is converted to at least one lower olefin having C₄-C₇ carbon atoms at a yield of at least about 80 wt. % of the maximum theoretical molar yield.

40. The process according to claim 21, wherein the process comprises the steps of:
a) feeding into a reaction vessel a stream comprising linear and/or branched C₅-C₈ primary alcohol which has at least one hydrogen atom attached to the carbon atom adjacent to the one bearing the alcohol group, at a concentration of 60-99 wt. %,
b) oxidation of the primary alcohol to the corresponding carboxylic acid, and
c) oxidative decarboxylation of the carboxylic acid to an olefin having one carbon atom less than the starting alcohol,
wherein linear or branched C₅-C₈ primary alcohol is converted to at least one lower olefin having C₄-C₇ carbon atoms at a yield of at least about 80 wt. % of the maximum theoretical molar yield.
